(19) United States
(12) Patent Application Publication
(10) Pub. No.: US 2024/0390525 A1
(43) Pub. Date: Nov. 28, 2024

(54) DEVICES AND METHODS FOR PRODUCING NEAR-INFRARED-II CONTRAST AGENT

(71) Applicant: Chung Yuan Christian University, Taoyuan City (TW)

(72) Inventors: Cheng-An LIN, Taoyuan City (TW); Chien-Liang LIU, Taoyuan City (TW); Yi-Tang SUN, Taoyuan City (TW)

(73) Assignee: Chung Yuan Christian University, Taoyuan City (TW)

(21) Appl. No.: 18/202,306

(22) Filed: May 26, 2023

(51) Int. Cl. A61K 49/00

(52) U.S. Cl. CPC A61K 49/0034; A61K 49/0056; A61K 49/0093

(57) ABSTRACT

Disclosed herein is a device for producing NIR-II contrast agents from fluorescent substances and serum albumin. The device comprises a first container, a mixing vessel, a first tube, and a first flow adjusting valve. According to the embodiments of the present disclosure, the first container and the mixing vessel are connected through the first tube, and the first flow adjusting valve is coupled to the first tube. Also disclosed herein are methods for producing the NIR-II contrast agents by using the present device.

### BACKGROUND OF THE INVENTION

### 1. Field of the Invention

The present disclosure relates to the preparation of near-infrared (NIR) fluorescent contrast agents. More particularly, the present disclosure relates to devices and methods for producing NIR-II contrast agents.

### 2. Description of Related Art

Contrast agents emitting near-infrared-I (NIR-I) fluorescence are widely used in biological detection and medical detection. However, the efficacy of the NIR-I emitting contrast agents is usually limited by tissue autofluorescence and photon scattering that interfere the image quality and the depth of tissue penetration.

It is recently reported that contrast agents emitting NIR-II fluorescence, which has a longer wavelength than NIR-I fluorescence, may overcome the defects of NIR-I fluorescence imaging. The NIR-II emitting contrast agents allows for deep tissue penetration, and provides micron-level spatial resolution and high signal-to-background ratio. NIR-II contrast agents have been regarded as the most promising in vivo fluorescence imaging technology.

However, those NIR-II contrast agents encounter the same problem with the traditional contrast agents, that is, they also need to be freshly prepared before use to achieve better imaging efficacy. For clinical use, imaging agents needs to be prepared in a bacteria-free room to avoid any contamination that greatly increases the lead time.

In view of the foregoing, there exists in the related art a need of a novel device for producing the NIR-II contrast agent in a more efficient manner.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The first aspect of the present disclosure is directed to a device for producing a NIR-II contrast agent from a fluorescent substance and a serum albumin. According to certain embodiments of the present disclosure, the device comprises a first container configured to house the fluorescent substance, a mixing vessel for receiving the serum albumin, a first tube arranged to connect the first container and the mixing vessel, and a first flow adjusting valve operably coupled to the first tube for controlling the flow rate of the fluorescent substance flowing out of the first container. In the embodiments, the first container is disposed vertically above the mixing vessel thereby allowing the fluorescent substance in the first container to flow therefrom, through the first tube and into the mixing vessel and mixed with the serum albumin to produce the NIR-II contrast agent. The thus-produced NIR-II contrast agent, when irradiated with a light having an excitation wavelength of 500-830 nm emits an enhanced NIR-II fluorescence at a wavelength ranging from 900 to 2,000 nm.

According to some embodiments of the present disclosure, the device comprises a mixing means disposed in the mixing vessel. In addition or alternatively, the device further comprises a first and second pressure adjusting valves respectively coupled to the first container and the mixing vessel for controlling respective pressures therein.

According to alternative embodiments of the present disclosure, the device further comprises a second container for housing the serum albumin, a second tube connecting the second container and the mixing vessel, and a second flow adjusting valve operably coupled to the second tube for controlling the flow rate of the serum albumin flow out of the second container. In the embodiments, the second container is disposed vertically above the mixing vessel thereby allowing the serum albumin in the second container to flow therefrom, through the second tube and into the mixing vessel. In some preferred embodiments, the device further comprises a third pressure adjusting valve coupled to the second container for controlling pressure therein.

According to certain embodiments of the present disclosure, the fluorescent substance is a cyanine dye, or a fluorescent nanoparticle. In some preferred embodiments of the present disclosure, the fluorescent substance is the cyanine dye. In one working example, the cyanine dye is clinically approved near-infrared dye indocyanine green (ICG). According to alternative embodiments of the present disclosure, the fluorescent substance is a fluorescent nanoparticle. Exemplary fluorescent nanoparticle is a fluorescent gold nanocluster or a silver sulfide quantum dot.

According to some embodiments, the serum albumin is human serum albumin (HSA) or bovine serum albumin (BSA).

The second aspect of the present disclosure is directed to a method for producing the said NIR-II contrast agent by use of one embodiment of the present device. According to some embodiments of the present disclosure, the method comprises steps of,
(a) providing the serum albumin to the mixing vessel;
(b) allowing the fluorescent substance in the first container to flow therefrom, through the first tube and into the mixing vessel and mix with the serum albumin the mixing vessel;
(c) adjusting the first flow adjusting valve to control the flow rate of the fluorescent substance flowing out from the first container; and
(d) mixing the content in the mixing vessel of step (c) at a speed of 120-1,200 rpm to produce the NIR-II contrast agent.

According to certain embodiments of the present disclosure, the fluorescent substance and the serum albumin are present in the NIR-II contrast reagent at a molar ratio of 1:1 to 1:500. Preferably, the fluorescent substance and the serum albumin are present in the NIR-II contrast reagent at a molar ratio of 1:1 to 1:256. In one working example, the fluorescent substance and the serum albumin are present in the NIR-II contrast reagent at a molar ratio of 1:2.

In some embodiments, the fluorescent substance is a cyanine dye, a fluorescent gold nanocluster, or a silver sulfide quantum dot, and the serum albumin is HSA or BSA.

The third aspect of the present disclosure is directed to a method for producing the said NIR-II contrast agent by use of another embodiment of the present device. According to certain embodiments of the present disclosure, the method comprises respectively adjusting the first and second flow adjusting valves to control the flow rates of the fluorescent substance and serum albumin respectively flowing out from the first and second containers and into the mixing vessel thereby producing the NIR-II contrast agent.

According to some embodiments of the present disclosure, the fluorescent substance and the serum albumin are present in the NIR-II contrast reagent at a molar ratio of 1:1 to 1:500. In some preferred embodiments, the fluorescent substance and the serum albumin are present in the NIR-II contrast reagent at a molar ratio of 1:1 to 1:256. According to one working example, the fluorescent substance and the serum albumin are present in the NIR-II contrast reagent at a molar ratio of 1:2.

According some exemplary embodiments of the present disclosure, the fluorescent substance is a cyanine dye, a fluorescent gold nanocluster, or a silver sulfide quantum dot, and the serum albumin is HSA or BSA.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram of the present device 100 according to one embodiment of the present disclosure; and

FIG. 2 is a schematic diagram of the present device 100 according to another embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

### DESCRIPTION OF THE INVENTION

The present disclosure aims at providing devices and methods of producing NIR-II contrast agents. Compared to existing devices and procedures, which must be carried out in a sterilized environment, the present device is easy and simple to use, in which the NIR-II contrast agents in produced in a closed system to eliminate the possibility of contamination without adversely affecting the purity and/or quality of the product.

### Devices for Producing NIR-II Contrast Agents

The first aspect of the present disclosure is directed to a device for producing NIR-II contrast agents from fluorescent substances and serum albumins. According to some embodiments of the present disclosure, after irradiating with a light having an excitation wavelength of 500-830 nm, the present contrast agent emits a NIR-II fluorescence at a wavelength ranging from 900 to 2,000 nm.

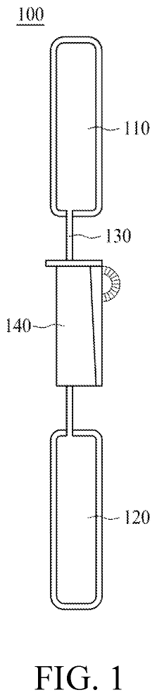

FIG. 1 is a schematic diagram depicting the device 100 according to one embodiment of the present disclosure. As illustrated, the device 100 comprises in its structure, a first container 110, a mixing vessel 120, a first tube 130, and a first flow adjusting valve 140.

The first container 110 is configured to house a first reactant, i.e., the fluorescent substance. Depending on desired purposes, the fluorescent substance may be a cyanine dye or a fluorescent nanoparticle. In some preferred embodiments, the fluorescent substance is a cyanine dye or its derivatives, for example, cyanine dye 3 (Cy3), Cy5, Cy7, indocyanine green (ICG), or the like. Preferably, the cyanine dye is ICG. Alternatively, the fluorescent substance is a fluorescent nanoparticle, for example, a fluorescent gold nanocluster or a silver sulfide quantum dot.

The mixing vessel 120 is configured to receive a second reactant, i.e., the serum albumin. According to some embodiments, the serum albumin may be derived from a bovine, a human, a monkey, a mouse, or a rat. Preferably, the serum albumin is human serum albumin (HSA) or bovine serum albumin (BSA).

In addition or alternatively, the device 100 further comprises first and second pressure adjusting valves respectively coupled to the first and second containers (110, 120) for controlling the respective pressures therein.

According to certain embodiments of the present disclosure, the fluorescent substance of the first container 110 is delivered to the mixing vessel 120 via the first tube 130, and mix with the serum albumin in the mixing vessel 120. To this purpose, a first flow adjusting valve 140 is operably coupled to the first tube 130 to control the flow rate of the fluorescent substance flowing out of the first container 110. Preferably, the first container 110 is disposed vertically above the mixing vessel 120, thereby allowing the fluorescent substance in the first container 100 to flow through the first tube 130 via gravity and into the mixing vessel 120. In addition, or alternatively, the mixing vessel 120 is equipped with a mixing means (e.g., a stirrer, mixing blades, and etc.) for mixing the content (e.g., fluorescent substance and serum albumin) therein.

The first container 110 and the mixing vessel 120 may be any receptacle known in the art for housing liquid, gas, or solid reactants. According to preferred embodiments, the fluorescent substance presents in the first container 110 is in liquid form. Depending on the intended purpose, the first container 110 and the mixing vessel 120 may be made of a translucent or opaque material. In some embodiments of the present disclosure, the first container 110 is made of an opaque material so as to protect the fluorescent substance from exposure to light. Examples of the material suitable for producing the first container 110 and the mixing vessel 120 include, but are not limited to, glasses, polymeric materials (e.g., polyethylene, poly vinyl chloride, polypropylene, polystyrene, and acrylonitrile butadiene styrene), or any material that does not react with the first and second reactants in the first container 110 and the mixing vessel 120. The thus-produced NIR-II contrast reagent is a complex of the fluorescent substance and serum albumin, in which the fluorescent substance is encapsulated within the serum albumin.

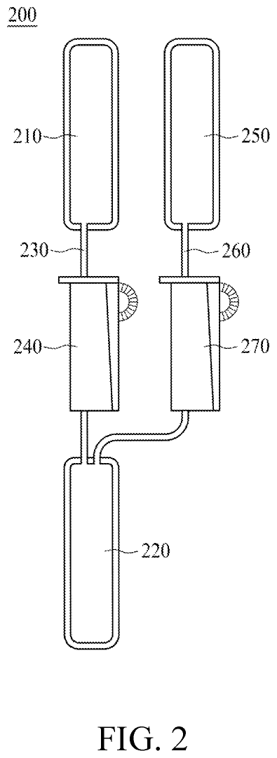

FIG. 2 is a schematic diagram depicting the device 200 according to another embodiment of the present disclosure. The structure of the device 200 is similar to that of the device 100 of FIG. 1, except the device 200 further comprises a second container 250, a second tube 260, and a second flow adjusting valve 270.

In this embodiment, the second container 250 is configured to house the serum albumin, which is disposed vertically above the mixing vessel 220. In this case, the serum albumin flows out from the second container 250 and drips into the mixing vessel 220 through the second tube 260 via gravity. Optionally, the flow rate of the serum albumin flowing out from the second container 250 may be controlled by the second flow adjusting valve 270, which is operably coupled to the second tube 260. In addition, or alternatively, the device 200 further comprises a third pressure adjusting valve coupled to the second container 250 to control the pressure therein.

Similar to the first container 110 and the mixing vessel 120 described above, the second container 250 may be any receptacle known in the art for housing liquid, gas, or solid reactants; and may be made of a translucent or opaque material described above.

### Methods for Producing the NIR-II Contrast Agent by Use of the Present Device

The second aspect of the present disclosure is directed to a method for producing the NIR-II contrast agent by using the device 100 of FIG. 1 or the device 200 of FIG. 2. According to preferred embodiments of the present disclosure, the method comprises steps of:
(a) providing the serum albumin to the mixing vessel;
(b) allowing the fluorescent substance in the first container to flow therefrom, through the first tube 130 and into the mixing vessel and mix with the serum albumin in the mixing vessel;
(c) adjusting the first flow adjusting valve to control the flow rate of the fluorescent substance flowing out from the first container 110; and
(d) mixing the content in the mixing vessel of step (c) at a speed of 120-1,200 rpm to produce the NIR-II contrast agent.

In step (a), the serum albumin is first provided to the mixing vessel 120. According to some embodiments, the serum albumin may be derived from a bovine, a human, a monkey, a mouse, or a rat. In some working examples, the serum albumin is HSA or BSA.

In step (b), the fluorescent substance contained in the first container 110 is dripped into the mixing vessel 120 via turning on the first flow adjusting valve 140, and then mixed with the serum albumin in the mixing vessel 120. According to some embodiments of the present disclosure, the fluorescent substance may be a cyanine dye or a fluorescent nanoparticle. In some preferred embodiments, the fluorescent substance is a cyanine dye or its derivatives, for example, the cyanine dye includes, but is not limited to, Cy3, Cy5, Cy7, ICG, IR-783, IRDye800, IR830, IR-E1050 or the like. Alternatively, the fluorescent substance is a fluorescent nanoparticle. In some embodiments, the fluorescent nanoparticle is a fluorescent gold nanocluster or a silver sulfide quantum dot.

For the purpose of forming the complex with desired structure, the number of molars of the serum albumin is preferably greater than that of the fluorescent substance during the reaction. Thus, in step (c), the fluorescent substance is delivered from the first container 110 to the mixing vessel 120 under a suitable flow rate via adjusting the first flow adjusting valve 140 so as to ensure the fluorescent substance is mixed with the serum albumin at a desired molar ratio.

In step (d), the fluorescent substance and the serum albumin present in the mixing vessel 120 of step (c) are mixed thoroughly to form the enhanced NIR-II contrast agent of the present disclosure. According to some embodiments of the present disclosure, the fluorescent substance and the serum albumin are mixed at a speed of 120-1,200 rpm. In one working example, a stirrer is disposed in the mixing vessel for the mixing purpose.

According to some preferred embodiments, the fluorescent substance and the serum albumin are present in the NIR-II contrast reagent at a molar ratio of 1:1 to 1:500. For example, the fluorescent substance and the serum albumin are present in the NIR-II contrast reagent at a molar ratio of 1:1, 1:2, 1:3, 1:4, 1:5, 1:6, 1:7, 1;8, 1:9, 1:10, 1:11, 1:12, 1:13, 1:14, 1:15, 1:16, 1:17, 1:18, 1:19, 1:20, 1:25, 1:30, 1:35, 1:40, 1:45, 1:50, 1:55, 1:60, 1:65, 1:70, 1:75, 1:80, 1:85, 1:90, 1:95, 1:100, 1:110, 1:120, 1:130, 1:140, 1:150, 1:160, 1:170, 1:180, 1:190, 1:200, 1:250, 255, 1:256, 1:257, 1:258, 1:259, 1:260, 1:265, 1:270, 1:275, 1:280, 1:285, 1:290, 1:295, 1:300, 1:350, 1:410, 1:450, or 1:500. Preferably, the molar ratio of the fluorescent substance and the serum albumin that is present in the NIR-II contrast reagent is ranging from 1:1 to 1:256. In one working example, the fluorescent substance and the serum albumin are present in the NIR-II contrast reagent at a molar ratio of 1:2.

The third aspect of the present disclosure is directed to a method for producing the NIR-II contrast agent by using the device 200 of FIG. 2. The method comprises respectively adjusting the first and second flow adjusting valves (240, 270) to control the flow rates of the fluorescent substance and serum albumin out from the first and second containers (210, 250) and into the mixing vessel 220 thereby producing the NIR-II contrast agent.

According to some preferred embodiments, the fluorescent substance is delivered from the first container 210 to the mixing vessel 220, and the serum albumin is delivered from the second container 250 to the mixing vessel 220.

### WORKING EXAMPLE

### Example 1 Preparation of NIR-II Contrast Agents

In the example, NIR-II contrast agents were produced by using the device of FIG. 1 or the device of FIG. 2.

### 1.1 NIR-II Contrast Agents Produced by the Device of FIG. 1.

The fluorescent substance ICG (having a concentration of 322.7 μM) and the HSA protein (having a concentration of 301.2 μM) were respectively provided to the first container and mixing vessel of the device of FIG. 1. The ICG solution was dripped into the mixing vessel at a flow rate of about 0.35-0.41 mL per minute and mixed with the HSA solution. Five NIR-II contrast agents, respectively designated as IH-1, IH-2, IH-3, IH-4, and IH-5, were prepared in accordance with the formulations summarized in Table 1.

TABLE 1
Formulations of five NIR-II contrast agents

| | | | Final | Final | Molar ratio of |
|---|---|---|---|---|---|
| NIR-II | ICG | HSA | concen- | concen- | ICG:HSA in |
| contrast | solution | solution | tration | tration | the NIR-II |
| agent | (322.7 μM) | (301.2 μM) | of ICG | of HSA | contrast agent |
| IH-1 | 3 mL | 3 mL | 161.4 | 150.1 | 1:0.9 |
| IH-2 | 2 mL | 4 mL | 107.6 | 200.8 | 1:1.9 |
| IH-3 | 1.5 mL | 4.5 mL | 80.7 | 225.9 | 1:3 |
| IH-4 | 1.5 mL | 6 mL | 64.6 | 241.0 | 1:4 |
| IH-5 | 1 mL | 5 mL | 53.8 | 251.0 | 1:5 |

### 1.2 NIR-II Contrast Agents Produced by the Device of FIG. 2

The fluorescent substance ICG (having a concentration of 322.7 μM) and the HSA protein (having a concentration of 301.2 μM) were respectively provided to the first container and second container of the device of FIG. 2. The ICG solution and the HSA solution were dripped into the mixing vessel. Five NIR-II contrast agents, respectively designated as IA-1, IA-2, IA-3, IA-4, and IA-5, were prepared in accordance with the formulations summarized in Table 2.

TABLE 2
Formulations of five NIR-II contrast agents

| | | | Final | Final | Molar ratio of |
|---|---|---|---|---|---|
| NIR-II | ICG | HSA | concen- | concen- | ICG:HSA in |
| contrast | solution | solution | tration | tration | the NIR-II |
| agent | (322.7 μM) | (301.2 μM) | of ICG | of HSA | contrast agent |
| IA-1 | 1 mL | 1 mL | 161.4 | 150.1 | 1:0.9 |
| IA-2 | 1 mL | 2 mL | 107.6 | 200.8 | 1:1.9 |
| IA-3 | 1 mL | 3 mL | 80.7 | 225.9 | 1:3 |
| IA-4 | 1 mL | 4 mL | 64.6 | 241.0 | 1:4 |
| IA-5 | 1 mL | 5 mL | 53.8 | 251.0 | 1:5 |

### Example 2 Characterization of the Present NIR-II Contrast Agents

The NIR-II contrast agents prepared in Example 1 (i.e. IH-1, IH-2, IH-3, IH-4, IH-5, IA-1, IA-2, IA-3, IA-4, and IA-5) were irradiated with light having an excitation wavelength of 780 nm, and the fluorescent signal emitted by the irradiated NIR-II contrast agents were detected by NIR Spectrometer. The ICG solution (without mixing with HSA) served as a control group in the study.

According to the results of Table 3, the NIR-II fluorescent intensity of the control (i.e. the ICG solution) was 214.9, while the NIR-II fluorescent intensities of IH-1, IH-2, IH-3, IH-4, IH-5, IA-1, IA-2, IA-3, IA-4, and IA-5 were respectively 576.0, 709.5, 657.5, 886.6, 852.5, 751.2, 619.7, 806.3, 766.0, and 857.9.

TABLE 3
NIR-II intensity of specified NIR-II contrast agents

| | NIR-II intensity* (a.u.)** |
|---|---|
| Control | 214.9 |
| IH-1 | 576.0 |
| IH-2 | 709.5 |
| IH-3 | 657.5 |
| IH-4 | 886.6 |
| IH-5 | 852.5 |
| IA-1 | 751.2 |
| IA-2 | 619.7 |
| IA-3 | 806.3 |
| IA-4 | 766.0 |
| IA-5 | 857.9 |

*Intensity: fluorescence counts detected at the wavelength ranging from 900 to 2,000 nm.
**a.u.: arbitrary units.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A device for producing a near-infrared-II (NIR-II) contrast agent from a fluorescent substance and a serum albumin, comprising:
a first container for housing the fluorescent substance;
a mixing vessel for receiving the serum albumin;
a first tube connecting the first container and the mixing vessel; and
a first flow adjusting valve operably coupled to the first tube for controlling the flow rate of the fluorescent substance flowing out of the first container;
wherein
the first container is disposed vertically above the mixing vessel thereby allowing the fluorescent substance in the first container to flow therefrom, through the first tube and into the mixing vessel, and mixed with the serum albumin to produce the NIR-II contrast agent; and
the NIR-II contrast agent, when irradiated with a light having an excitation wavelength of 500-830 nm emits a NIR-II fluorescence at a wavelength ranging from 900 to 2,000 nm.

2. The device of claim 1, further comprising,
a second container for housing the serum albumin;
a second tube connecting the second container and the mixing vessel; and
a second flow adjusting valve operably coupled to the second tube for controlling the flow rate of the serum albumin flowing out of the second container;
wherein
the second container is disposed vertically above the mixing vessel thereby allowing the serum albumin in the second container to flow therefrom, through the second tube and into the mixing vessel.

3. The device of claim 1, further comprising a mixing means disposed in the mixing vessel.

4. The device of claim 1, further comprising a first and second pressure adjusting valves respectively coupled to the first container and the mixing vessel for controlling respective pressures therein.

5. The device of claim 2, further comprising a third pressure adjusting valve coupled to the second container for controlling pressure therein.

6. The device of claim 1, wherein the fluorescent substance is a cyanine dye, or a fluorescent nanoparticle.

7. The device of claim 6, wherein the fluorescent substance is the cyanine dye.

8. The device of claim 7, wherein the cyanine dye is indocyanine green (ICG).

9. The device of claim 8, wherein the fluorescent substance is the fluorescent nanoparticle, and
the fluorescent nanoparticle is a fluorescent gold nanocluster or a silver sulfide quantum dot.

10. The device of claim 1, the serum albumin is a human serum albumin (HSA) or a bovine serum albumin (BSA).

11. A method for producing a near-infrared-II (NIR-II) contrast agent by use of the device of claim 1, comprising,
(a) providing the serum albumin to the mixing vessel;
(b) allowing the fluorescent substance in the first container to flow therefrom, through the first tube and into the mixing vessel and mix with the serum albumin in the mixing vessel;
(c) adjusting the first flow adjusting valve to control the flow rate of the fluorescent substance flowing out from the first container; and
(d) mixing the content in the mixing vessel of step (c) at a speed of 120-1,200 rpm to produce the NIR-II contrast agent.

12. The method of claim 11, wherein the fluorescent substance and the serum albumin are present in the NIR-II contrast reagent at a molar ratio of 1:1 to 1:500.

13. The method of claim 12, wherein the fluorescent substance and the serum albumin are present in the NIR-II contrast reagent at a molar ratio of 1:1 to 1:256.

14. The method of claim 13, wherein the fluorescent substance and the serum albumin respectively housed in the first and second containers are present in a molar ratio of 1:2.

15. The method of claim 11, wherein the fluorescent substance is a cyanine dye, a fluorescent gold nanocluster or a silver sulfide quantum dot, and the serum albumin is a human serum albumin (HSA) or a bovine serum albumin (BSA).

16. A method for producing a near-infrared-II (NIR-II) contrast agent by use of the device of claim 2, comprising respectively adjusting the first and second flow adjusting valves to control the flow rates of the fluorescent substance and serum albumin respectively flowing out from the first and second containers and into the mixing vessel thereby producing the NIR-II contrast agent.

17. The method of claim 16, wherein the fluorescent substance and the serum albumin are present in the NIR-II contrast reagent at a molar ratio of 1:1 to 1:500.

18. The method of claim 17, wherein the fluorescent substance and the serum albumin are present in the NIR-II contrast reagent at a molar ratio of 1:1 to 1:256.

19. The method of claim 18, wherein the fluorescent substance and the serum albumin respectively housed in the first and second containers are present in a molar ratio of 1:2.

20. The method of claim 16, wherein the fluorescent substance is a cyanine dye, a fluorescent gold nanocluster or a silver sulfide quantum dot, and the serum albumin is a human serum albumin (HSA) or a bovine serum albumin (BSA).
